(19) United States
(12) Patent Application Publication
(10) Pub. No.: US 2026/0260343 A1
(43) Pub. Date: Sep. 3, 2026

(54) MAGNETIC RESONANCE IMAGING RADIOMICS FOR MANAGEMENT OF PANCREATIC CANCER AND ITS PRECURSORS

(71) Applicant: H. LEE MOFFITT CANCER CENTER AND RESEARCH INSTITUTE, INC., Tampa, FL (US)

(72) Inventors: Jennifer B. PERMUTH, Tampa, FL (US); Daniel JEONG, Wesley Chapel, FL (US)

(21) Appl. No.: 19/491,246

(22) PCT Filed: Jun. 3, 2024
(86) PCT No.: PCT/US2024/032200

§ 371 Date: Dec. 8, 2025

Related U.S. Application Data
(60) Provisional application No. 63/507,450, filed on Jun. 10, 2023.

(51) Int. Cl. G06T 7/00; G06T 7/45; G16H 20/40; G16H 30/40; G16H 50/20; G16H 70/60

(52) U.S. Cl. CPC G06T 7/0012; G06T 7/45; G16H 20/40; G16H 30/40; G16H 50/20; G16H 70/60; G06T 2207/10088; G06T 2207/30096

(57) ABSTRACT

A device may obtain a magnetic resonance (MR) image of a region of interest (ROI) that includes at least a portion of the pancreatic lesion of the subject. A device may extract one or more radiomic features from the ROI of the MR image. A device may calculate a score from the one or more extracted radiomic features. A device may compare the score to a reference value. A device may classify the pancreatic lesion as malignant or benign based on the comparison.

### CROSS-REFERENCE TO RELATED APPLICATIONS

This application claims the benefit of U.S. provisional patent application No. 63/507,450, filed on Jun. 10, 2023, and titled “MAGNETIC RESONANCE IMAGING RADIOMICS FOR MANAGEMENT OF PANCREATIC CANCER AND ITS PRECURSORS,” the disclosure of which is expressly incorporated herein by reference in its entirety.

### STATEMENT REGARDING FEDERALLY FUNDED RESEARCH

This invention was made with government support under Grant No. R37CA229810 awarded by the National Institutes of Health. The government has certain rights in the invention.

### BACKGROUND

The era of big data has transformed radiologic images from simple digital pictures into a trove of quantitative information. Radiomics is defined as the high-throughput extraction and analysis of quantitative features from digital medical images to further understanding of the microenvironments that harbor disease [1,2]. The prospect of uncovering a predictive model using standard-of-care images, laboratory and genomic assays, and other patient characteristics to distinguish precursor tumors and mitigate the morbidity and mortality associated with cancer has garnered considerable interest. In particular, intraductal papillary mucinous neoplasms (IPMNs) present a unique challenge and an interesting target for the potential of clinical radiomics.

Pancreatic ductal adenocarcinoma (PDAC) is one of the most aggressive cancers worldwide, with a five-year survival rate of about 5-10% [3]. The majority of cases present with late-stage disease due to the lack of early detection strategies [2,4]. 20-30% of pancreatic adenocarcinomas may arise from IPMNs, noninvasive cystic precursor lesions that can progress from low-to-high-grade dysplasia and eventually to invasive carcinoma [4-6]. The challenge is that while IPMNs are currently one of the only radiographically identifiable precursors of pancreatic cancer, and IPMNs account for almost half of pancreatic cysts found in computed tomography (CT) scans and magnetic resonance imaging (MRI) studies each year, only a third of resected IPMNs are associated with invasive carcinoma [2,4,7]. Patients with non-invasive IPMNs have a 5-year survival rate of 90-100% following resection, yet pancreatic resection has been associated with an operative mortality of 2-4% and morbidity of 40-50% [2,8,9]. Consequently, there is a critical need to accurately determine the malignant potential of IPMNs from imaging to be able to balance the risk of malignant transformation with the risk of pancreatic resection [7].

The internationally accepted, consensus-based 2012 Fukuoka (ICG) guidelines regarding prediction of high-grade dysplasia and invasive carcinoma, surveillance, and postoperative management of IPMNs were revised in 2017 but remain inadequate [8,10]. IPMNs may involve the main pancreatic duct (MD-IPMN), branch duct (BD-IPMN), or both (mixed-type IPMN) and may be classified into four pathologic subtypes: gastric, intestinal, pancreatobiliary, and oncocytic [6,7]. Current recommendations support active surgical treatment for main duct and mixed type IPMNs, and rely heavily on qualitative metrics to identify “high-risk stigmata” or “worrisome features” which are used to predict pathology and advise clinical management [5,7,9]. However, several studies have determined these guidelines continue to lead to unnecessary surgeries for benign lesions [2,7-9,11].

### SUMMARY

In view of the unnecessary surgeries conducted for benign pancreatic lesions, described herein are techniques that use magnetic resonance imaging (MRI) radiomics to enable a reliable evaluation of cystic pancreatic lesions such as intraductal papillary mucinous neoplasms (IPMNs) to discern malignant IPMNs from benign IPMNs. Thus, the feasibility of using radiomics to improve pre-operative patient risk stratification by comparing MRI radiomics features with “gold standard” pathology is shown herein.

A first aspect concerns a method for evaluating the malignant potential of a pancreatic lesion, such as an IPMN, using MRI radiomic features capable of distinguishing malignant lesions from benign lesions. Another aspect concerns a method of treatment of IPMNs that have been classified as malignant based at least in part on the evaluation method described herein. Another aspect concerns a computer-readable storage device storing computer executable instructions that, when executed, control a processor to perform operations, including steps for the evaluating the malignant potential of a pancreatic lesion.

In some aspects, the techniques described herein relate to a method for evaluating the malignant potential of a pancreatic lesion of a subject, the method including: obtaining a magnetic resonance (MR) image of a region of interest (ROI) that includes at least a portion of the pancreatic lesion of the subject; extracting one or more radiomic features from the ROI of the MR image; calculating a score from the one or more extracted radiomic features; comparing the score to a reference value; and classifying the pancreatic lesion as malignant or benign based on the comparison.

In some aspects, the techniques described herein relate to a method, wherein the pancreatic lesion is an intraductal papillary mucinous neoplasm (IPMN).

In some aspects, the techniques described herein relate to a method, wherein the one or more radiomic features include one or more T1 weighted radiomic features.

In some aspects, the techniques described herein relate to a method, wherein the one or more radiomic features include one or more T2 weighted radiomic features.

In some aspects, the techniques described herein relate to a method, wherein the one or more radiomic features include a plurality of radiomic features including one or more T1 weighted radiomic features, and one or more T2 weighted radiomic features.

In some aspects, the techniques described herein relate to a method, wherein the one or more radiomic features include at least one feature within the domain of texture analysis.

In some aspects, the techniques described herein relate to a method, wherein the one or more radiomic features include at least one gray level co-occurrence matrix (GLCM) feature.

In some aspects, the techniques described herein relate to a method, wherein the one or more radiomic features include at least one radiomic feature from those listed in Table 1, or at least one radiomic feature listed in Table 2, or a plurality of radiomic features that includes one or more radiomic features from those listed in Table 1 and includes one or more radiomic features from those listed in Table 2.

In some aspects, the techniques described herein relate to a method, wherein the MR image is a set of MR images including: T1W pre-contrast images acquired by gradient echo with fat saturation, or T2W non-fat saturated images acquired by fast spin-echo, or both.

In some aspects, the techniques described herein relate to a method, wherein said extracting of one or more radiomic features includes contouring the ROI on pre-operative abdominal axial T1 weighted fat saturated pre-contrast sequences, or T2 weighted echo-planar fast spin echo (single-shot) sequences, or both.

In some aspects, the techniques described herein relate to a method, wherein said extracting of one or more radiomic features includes performing whole lesion semi-automated tumor segmentation to generate a three-dimensional volume of interest.

In some aspects, the techniques described herein relate to a method, wherein the method further includes pre-normalization of the MR image, prior to said extracting of one or more radiomic features, to provide one or more of: normalize z-score, provide gray-level discretization, and voxel size resampling.

In some aspects, the techniques described herein relate to a method for treating an intraductal papillary mucinous neoplasm (IPMN) in a subject, including administering a treatment for the IPMN to the subject, wherein the subject has previously been identified as having the IPMN, and wherein the IPMN as been classified as malignant using the techniques described herein.

In some aspects, the techniques described herein relate to a method, wherein the treatment includes surgical resection, pancreatoduodenectomy (Whipple procedure), chemotherapy, radiation, immunotherapy, or a combination of two or more of the foregoing.

In some aspects, the techniques described herein relate to a method, wherein the treatment includes surgical resection.

In some aspects, the techniques described herein relate to a non-transitory computer-readable storage device for evaluating the malignant potential of a pancreatic lesion of a subject, the non-transitory computer-readable storage device storing computer executable instructions that, when executed, control a processor to perform operations, the operations including: accessing a magnetic resonance (MR) image of a region of interest (ROI) that includes at least a portion of the pancreatic lesion of the subject; extracting one or more radiomic features from the ROT of the MR image; calculating a score from the one or more extracted radiomic features; comparing the score to a reference value; and classifying the pancreatic lesion as malignant or benign based on the comparison.

In some aspects, the techniques described herein relate to a non-transitory computer-readable storage device, wherein said accessing includes retrieving or otherwise acquiring electronic data from a computer memory, receiving a computer file over a computer network.

In some aspects, the techniques described herein relate to a non-transitory computer-readable storage device, wherein the pancreatic lesion is an intraductal papillary mucinous neoplasm (IPMN).

In some aspects, the techniques described herein relate to a non-transitory computer-readable storage device, wherein the one or more radiomic features include one or more T1 weighted radiomic features.

In some aspects, the techniques described herein relate to a non-transitory computer-readable storage device, wherein the one or more radiomic features include one or more T2 weighted radiomic features.

In some aspects, the techniques described herein relate to a non-transitory computer-readable storage device, wherein the one or more radiomic features include a plurality of radiomic features including one or more T1 weighted radiomic features, and one or more T2 weighted radiomic features.

In some aspects, the techniques described herein relate to a non-transitory computer-readable storage device, wherein the one or more radiomic features include at least one feature within the domain of texture analysis.

In some aspects, the techniques described herein relate to a non-transitory computer-readable storage device, wherein the one or more radiomic features include at least one gray level co-occurrence matrix (GLCM) feature.

In some aspects, the techniques described herein relate to a non-transitory computer-readable storage device, wherein the one or more radiomic features include at least one radiomic feature from those listed in Table 1, or at least one radiomic feature listed in Table 2, or a plurality of radiomic features that includes one or more radiomic features from those listed in Table 1 and includes one or more radiomic features from those listed in Table 2.

In some aspects, the techniques described herein relate to a non-transitory computer-readable storage device, wherein the MR image is a set of MR images including: T1W pre-contrast images acquired by gradient echo with fat saturation, or T2W non-fat saturated images acquired by fast spin-echo, or both.

In some aspects, the techniques described herein relate to a non-transitory computer-readable storage device, wherein said extracting of one or more radiomic features includes contouring the ROI on pre-operative abdominal axial T1 weighted fat saturated pre-contrast sequences, or T2 weighted echo-planar fast spin echo (single-shot) sequences, or both.

In some aspects, the techniques described herein relate to a non-transitory computer-readable storage device, wherein said extracting of one or more radiomic features includes performing whole lesion semi-automated tumor segmentation to generate a three-dimensional volume of interest.

In some aspects, the techniques described herein relate to a non-transitory computer-readable storage device, wherein the pre-normalization of the MR image, prior to said extracting of one or more radiomic features, to provide one or more of: normalize z-score, provide gray-level discretization, and voxel size resampling.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 shows an outline of the current (Sendai 2012) consensus guidelines for IPMN management.

FIG. 2 shows a flow chart summarizing the clinical management of pancreatic cysts.

FIG. 3 is a flow chart illustrating example operations for evaluating the malignant potential of a pancreatic lesion of a subject according to implementations described herein.

FIG. 4 is an example computing device.

### DETAILED DESCRIPTION

The techniques described herein endeavor to evaluate pre-operative magnetic resonance imaging-based (MRI-based) radiomics characteristics in comparison to surgical pathology in patients with an intraductal papillary mucinous neoplasms (IPMN) with the objective to identify quantitative features that could distinguish malignant histology. As described herein, MRI-based radiomics features may be used to predict malignant histology and may be used to improve current guidelines for clinical management for patients with IPMNs.

In Example 1, the feasibility of using MRI-based radiomics as markers to predict the malignant potential of IPMNs is demonstrated. The results described herein demonstrate 14 features on T1 weighted MRI sequences and 10 features on T2 weighted MRI sequences which were able to discriminate malignant from benign IPMNs. While previous research has demonstrated that models combining CT-based radiomics with traditional guidelines improve the predictive performance for diagnosing malignant IPMNs, this study compares pre-operative MRI radiomics with gold standard post-operative pathology.

As described herein, pre-operative MRI-based radiomics characteristics have been evaluated in comparison to surgical pathology in patients with IPMN with the objective to identify qualitative features that could distinguish malignant histology from benign histology. The techniques described herein show that MRI-based radiomics features may be used to predict malignant histology and may be used to improve current guidelines for clinical management for patients with IPMNs.

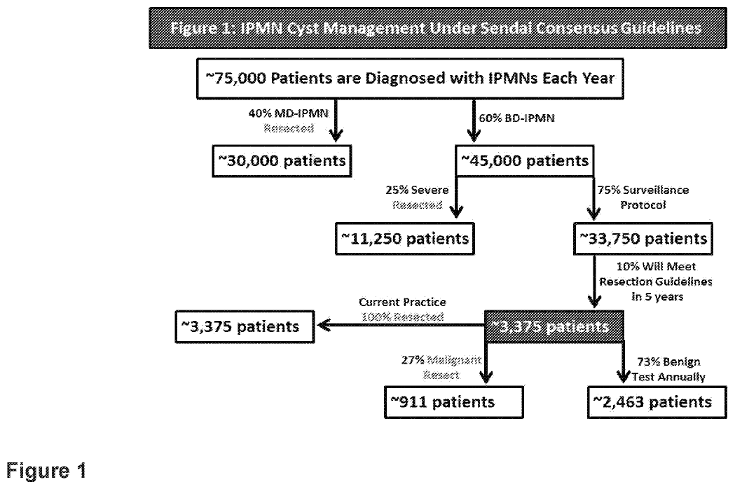

FIG. 1 shows an outline of the current (Sendai 2012) consensus guidelines for IPMN management. There are greater than 75,000 IPMNs detected incidentally in the general population each year due to increased computer tomography and (CT) and magnetic resonance imaging (MRI).

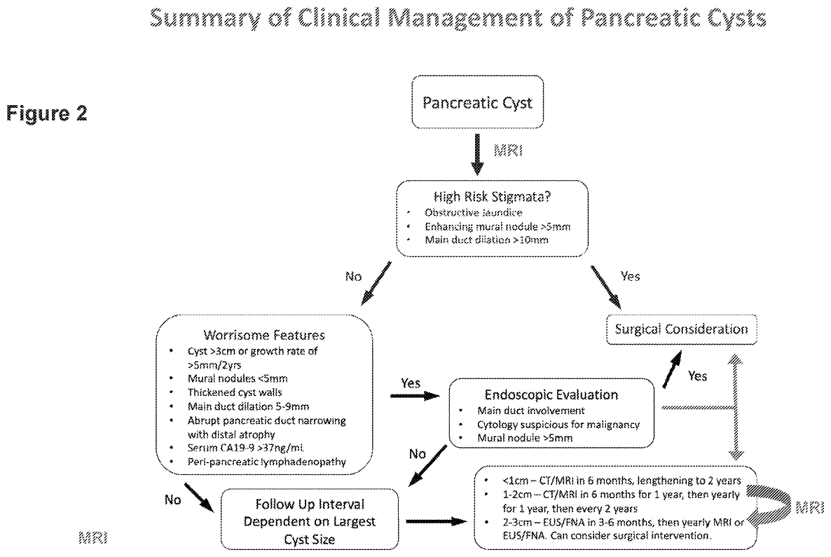

FIG. 2 shows a flow chart summarizing the clinical management of pancreatic cysts, including some opportunities where the techniques described herein may be utilized to evaluate the malignant potential of a cystic pancreatic lesion.

In one aspect, a method for evaluating the malignant potential of a pancreatic lesion such as an IPMN is described. The method comprises:
obtaining a magnetic resonance (MR) image of a region of interest (ROI) that includes at least a portion of the pancreatic lesion of the subject;
extracting one or more radiomic features from the ROI of the MR image;
calculating a score from the one or more extracted radiomic features;
comparing the score to a statistically derived reference value; and
classifying the pancreatic lesion as malignant or benign based on the comparison.

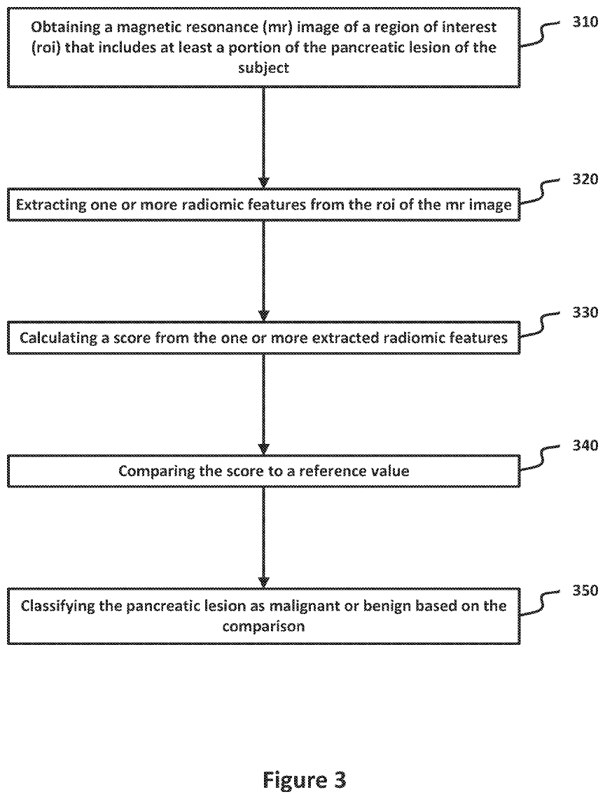

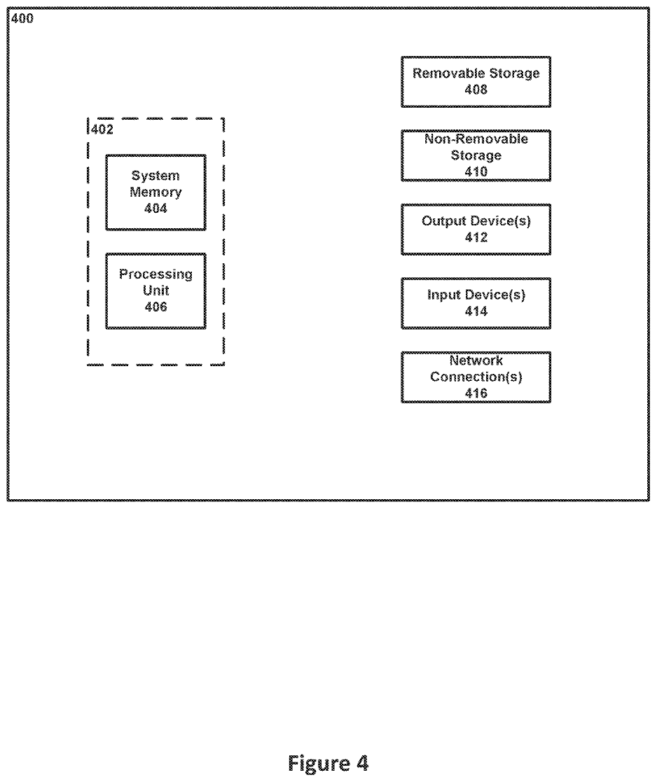

For example, FIG. 3 is a flowchart of an example method for evaluating the malignant potential of a pancreatic lesion of a subject. This disclosure contemplates that logical operations can be performed using a computing device such as the computing device 400 of FIG. 4.

At step 310, a magnetic resonance (MR) image of a region of interest (ROI) that includes at least a portion of the pancreatic lesion of the subject is obtained.

Obtaining the MR image may comprise accessing the MR image. Accessing the MR image(s) may include retrieving or otherwise acquiring electronic data from a computer memory, receiving a computer file over a computer network, or other computer or electronic based action. Each MR image, which may be a member of a set of MR images, has a plurality of pixels, a pixel having an intensity.

At step 320, one or more radiomic features from the ROI of the MR image are extracted.

The number of radiomic features extracted from the ROI of the MR image(s) may be 1, 2, 3, 4, 5, 6, 7, 8, 9, 10, 11, 12, 13, 14, or more. In some embodiments, the one or more radiomic features comprise one or more T1 weighted radiomic features. In some embodiments, the one or more radiomic features comprise one or more T2 weighted radiomic features. In some embodiments, the one or more radiomic features comprise a plurality of radiomic features including one or more T1 weighted radiomic features, and one or more T2 weighted radiomic features.

Two basic types of MR images are T1-weighted and T2-weighted images, often referred to as T1 and T2 images. Each tissue returns to its equilibrium state after excitation by the independent relaxation processes of T1 (spin-lattice; that is, magnetization in the same direction as the static magnetic field) and T2 (spin-spin; transverse to the static magnetic field). To create a T1-weighted image, magnetization is allowed to recover before measuring the MR signal by changing the repetition tine (TR). To create a T2-weighted image, magnetization is allowed to decay before measuring the MR signal by changing the echo time (TE).

The timing of radiofrequency pulse sequences used to make T1 images results in images which highlight fat tissue within the body. The timing of radiofrequency pulse sequences used to make T2 images results in images which highlight fat and water within the body. In some embodiments, the MR image(s) includes a T1-weighted image. In some embodiments, the MR image(s) includes a T2-weighted image. In some embodiments, the MR image(s) is a plurality of MR images that includes at least one T1-weighted image and at least one T2-weighted image. An MRI sequence in MRI is a particular setting of pulse sequences and pulsed field gradients, resulting in a particular image appearance.

In some embodiments, the one or more radiomic features comprise at least one feature within the domain of texture analysis. In some embodiments, the one or more radiomic features comprise at least one gray level co-occurrence matrix (GLCM) feature. In some embodiments, the one or more radiomic features comprise at least one radiomic feature from those listed in Table 1, or at least one radiomic feature listed in Table 2, or a plurality of radiomic features that includes one or more radiomic features from those listed in Table 1 and includes one or more radiomic features from those listed in Table 2.

In some embodiments, the MR image is a set of MR images including: T1W pre-contrast images acquired by gradient echo with fat saturation, or T2W non-fat saturated images acquired by fast spin-echo, or both.

In some embodiments, the extracting of one or more radiomic features includes contouring the ROI on pre-operative abdominal axial T1 weighted fat saturated pre-contrast sequences, or T2 weighted echo-planar fast spin echo (single-shot) sequences, or both.

In some embodiments, the extracting of one or more radiomic features includes performing whole lesion semi-automated tumor segmentation to generate a three-dimensional volume of interest.

In some embodiments, the method further includes the pre-normalization of the MR image, prior to said extracting of one or more radiomic features, to provide one or more of the following: normalize z-score, provide gray-level discretization, and voxel size resampling.

At step 330, a score is calculated from the one or more extracted radiomic features.

At step 340, the score is compared to a reference value.

At step 350, the pancreatic lesion is classified as malignant or benign based on the comparison.

Another aspect concerns a method for treating IPMN in a subject, comprising administering a treatment for the IPMN to the subject, wherein the subject has previously been identified as having the IPMN, and wherein the IPMN as been classified as malignant using the evaluation method described herein. Examples of treatments for the malignant IPMN include, but are not limited to, surgical resection, pancreatoduodenectomy (Whipple procedure), chemotherapy, radiation, immunotherapy, or a combination of two or more of the foregoing.

In the treatments methods involving administering a treatment to the subject, the treatment may be, for example, surgery (e.g., resection), radiation, and/or administration of an anti-cancer agent such as a chemotherapuetics (e.g., DNA-binding alkylating agents) and immune modulators (see, for example, Wong K K et al., “The Role of Precision Medicine in Pancreatic Cancer: Challenges of Targeted Therapy, Immune Modulating Treatment, Early Detection, and Less Invasive Operations,” Cancer Transl Med., 2:41-7, 2016; Wolfgang C L et al., “Recent Progress in Pancreatic Cancer,” CA Cancer J Clin, 63(5):318-348, September 2013; Grutzman R et al., “Intraductal Papillary Mucinous Neoplasia (IPMN) of the Pancrease, its Diagnosis, Treatment and Prognosis,” Dtsch Arztebl Int., 108:46:788-794, November 2011; Grutzman R et al. “Intraductal Papillary Mucinous Tumours of the Pancreas: Biology, Diagnosis, and Treatment, The Oncologist, 15:1294-1309, 2010, which are each incorporated herein by reference in their entirety).

Examples of interventions for pancreatic cancer and lesions such as IPMNs, which may be utilized as potential treatments with the methods described herein, include but are not limited to:

### Surgeries (Surgical Interventions):

Enucleation (removing just the tumor): If a pancreatic neuroendocrine tumor is small, just the tumor itself is removed. This is called enucleation.

Whipple procedure (pancreaticoduodenectomy): removing the head of the pancreas and sometimes the body of the pancreas as well. Nearby structures such as part of the small intestine, part of the bile duct, the gallbladder, lymph nodes near the pancreas, and sometimes part of the stomach are also removed.

Distal pancreatectomy: removing only the tail of the pancreas or the tail and a portion of the body of the pancreas.

Total pancreatectomy: removing the entire pancreas, as well as the gallbladder, part of the stomach and small intestine, and the spleen.

Palliative surgery: If the cancer has spread too far to be removed completely, any surgery being considered would be palliative (intended to relieve or prevent symptoms).

### Ablative Treatments:

Ablation refers to treatments that destroy tumors, usually with extreme heat or cold.

Radiofrequency ablation (RFA): Using high-energy radio waves for treatment. A thin, needle-like probe is placed through the skin and into the tumor. An electric current then passes through the tip of the probe, which heats the tumor and destroys the cancer cells. This treatment is used mainly for small tumors.

Microwave thermotherapy: This procedure is similar to RFA, except microwaves are used to heat and destroy the tumor.

Cryosurgery (also known as cryotherapy or cryoablation): Destroying a tumor by freezing it. A thin metal probe is guided into the tumor, and very cold gasses pass through the probe to freeze the tumor, killing the cancer cells.

Embolization: Embolization involves injecting substances into an artery to try to block the blood flow to cancer cells, causing them to die.

Arterial embolization: This is also known as trans-arterial embolization (or TAE). A catheter (a thin, flexible tube) is put into an artery through a small cut in the inner thigh and threaded up into the artery feeding the tumor and small particles are injected into the artery to plug it up.

Chemoembolization: This is also known as trans-arterial chemoembolization (or TACE), which combines embolization with chemotherapy. This involves using tiny beads that give off a chemotherapy drug for the embolization. TACE can also be done by giving chemotherapy through the catheter directly into the artery, then plugging up the artery.

Radioembolization: This combines embolization with radiation therapy. This involves injecting small radioactive beads (called microspheres) into the artery. The beads lodge in the blood vessels near the tumor, where they give off small amounts of radiation to the tumor site for several days. The radiation travels a very short distance, so its effects are limited mainly to the tumor.

### Anti-Cancer Agents Such as Drugs:

Abraxane (Paclitaxel Albumin-stabilized Nanoparticle Formulation)
Afinitor (Everolimus)
Erlotinib Hydrochloride
Everolimus
Fluorouracil Injection
Gemcitabine Hydrochloride
Mitomycin C
Mitozytrex (Mitomycin C)
Mutamycin (Mitomycin C)
Sunitinib Malate
Sutent (Sunitinib Malate)
Lanreotide Acetate
Lutathera (Lutetium Lu 177-Dotatate)
Lutetium Lu 177-Dotatate
Somatuline Depot (Lanreotide Acetate)
Folfirinox: FOL=Leucovorin Calcium (Folinic Acid); F=Fluorouracil; IRIN=Irinotecan Hydrochloride; OX=Oxaliplatin (this is different from what you identified below)
Gemcitabine Hydrochloride and Cisplatin
Gemcitabine Hydrochloride and Oxaliplatin
OFF: O=Oxaliplatin; F=Fluorouracil; and F=Leucovorin Calcium (Folinic Acid)

Examples of chemotherapeutic treatment for pancreatic cancer and lesions such as IPMNs include, but are not limited to: erlotinib, Gemcitabine (Gemzar), 5-fluorouracil (5-FU), Irinotecan (Camptosar), Oxaliplatin (Eloxatin), Albumin-bound paclitaxel (Abraxane), Capecitabine (Xeloda), Cisplatin, Paclitaxel (Taxol), Docetaxel (Taxotere), and Irinotecan liposome (Onivyde). Examples of combination treatments include: a combination of Albumin-bound paclitaxel and gemcitabine; combination of Irinotecan liposome, 5-FU, and folinic acid (leucovorin); and combination of 5-FU, irinotecan and oxaliplatin (Folfirinox).

As used herein, the term “(therapeutically) effective amount” refers to an amount of an agent (e.g., an anti-cancer agent) effective to treat a disease or disorder in a mammal, such as pancreatic cancer or an IPMN. In the case of cancer, the therapeutically effective amount of the agent may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the administered agent prevents growth of and/or kills existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the anti-cancer agent refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

As used herein, the term “anti-cancer agent” refers to a substance or treatment (e.g., radiation therapy) that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY).

As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, TN), and anti-androgens such as flutamide, nilutamide, bicalutamide, leuprolide, and goserelin, etc. In some embodiments, the chemotherapeutic agent is one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

As used herein, the term “subject” is defined herein to include animals such as mammals, including, but not limited to, primates (e.g., humans), cows, sheep, goats, horses, dogs, cats, rabbits, rats, mice and the like. In some embodiments, the subject is a human.

In another aspect, a computer-readable storage device may store computer executable instructions that, if executed by a machine (e.g., computer, processor), cause the machine to perform the methods described above. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a solid state device (SSD), a memory stick, a data storage device, and other media from which a computer, a processor, on the cloud in a SAS mode or other electronic device can read.

By displaying the annotated image of the ROI, the classification, or the features, the apparatus described herein provides a timely and intuitive way for a human pathologist, a personalized cancer treatment system, or a diagnostic system to predict the malignant potential of a cystic pancreatic lesion such as an IPMN.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

As used herein, the term “tumor” refers to all neoplastic cell growth and proliferation, whether malignant or benign, and all pre-cancerous and cancerous cells and tissues. For example, a particular cancer may be characterized by a solid tumor mass or a non-solid tumor. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture, or weight of the tissue. However, some primary tumors are not palpable and can be detected only through medical imaging techniques such as X-rays (e.g., mammography), or by needle aspirations. The use of these latter techniques is more common in early detection. Depending upon the agent, anti-cancer agents can be administered locally at the site of a tumor (e.g., by direct injection) or remotely.

As used in this specification, the singular forms “a”, “an”, and “the” include plural reference unless the context clearly dictates otherwise. Thus, for example, a reference to “an image” includes one or more images. A reference to “a radiomic feature” includes one or more radiomic features, and so forth.

The practice of the methods described herein can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

### Example Computing Device

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by box 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for.

### Materials and Methods

Pre-operative MRI studies were evaluated for a retrospective single center cohort of forty-three subjects who had surgical resection at a tertiary cancer center with histology demonstrating intraductal papillary mucinous neoplasm. Institutional Review Board approval was obtained and waiver of consent was granted. Data was acquired in compliance with all applicable Health Insurance Portability and Accountability Act (HIPAA) regulations, and research was performed in accordance with the Declaration of Helsinki principles. Subjects had surgical resection between 2007 and 2016 and preoperative MRI available within three months preceding surgery, while subjects who had inaccessible pathology reports were excluded. The initial cohort was drawn from a database search of radiological records with matching pathology result availability. Subjects who had surgical pathology grade demonstrating low or moderate dysplasia were considered “benign”, while surgical grade demonstrating high grade dysplasia or invasive carcinoma were considered “malignant”. Clinical characteristics including demographic information, symptoms, and laboratory data were also obtained by review of medical records.

MRI Acquisition. All magnetic resonance (MR) examinations were performed on 1.5T MRI scanners: n=34 (Siemens Medical Solutions, Ehrlangen, Germany); n=5 (General Electric Medical Systems, Waukesha, WI, USA); n=3 (Philips Medical Systems, Amsterdam, Netherlands); n=1 (Toshiba-MEC, Tokyo, Japan) using standard pulse sequences. T1W precontrast images were acquired by gradient echo with fat saturation {Echo time (TE)=1-5 ms, repetition time (TR)−3-304 ms, flip angle=10-80 ms, slice thickness=3-7 mm, field of view (FOV)=[300-460]×[200=420] mm², matrix=[256-512]×[160-512], number of excitations/averages (NEX)=1, in-plane pixel resolution 0.59-1.80 mm}. T2W non-fat saturated images were acquired by fast spin-echo [effective TE=58-184 ms, TR=452-24,000 ms, flip angle=90-180 ms, slice thickness=4-9 mm, FOV=[300-450]×[194-430] mm², matrix=[512-256]×[192-512], NEX (averages)=1-2, in-plane pixel resolution 0.59-1.76 mm].

Histopathological Data. Hematoxylin and eosin-stained slides of resected tumors were reviewed by a dedicated oncologic pathologist. Pathologic variables including tumor size, grade, and location were recorded.

Tumor Segmentation and Radiomics analysis. To extract radiomics features, regions of interest (ROIs) were contoured on pre-operative abdominal axial T1 weighted fat saturated pre-contrast and T2 weighted echo-planar fast spin echo (single-shot) sequences. On each series, whole lesion semi-automated tumor segmentation was performed by an experienced oncologic radiologist on every axial image containing the pancreatic lesion using Healthmyne (Healthmyne Inc, Madison, WI, USA) software to generate a three-dimensional volume of interest. Data reduction was obtained by univariate analysis to extract 156 relevant, unique, and non-correlating size, shape and textural features while absolute MRI signal intensity related features were excluded. Prior to feature extraction, prenormalization of MRI images was performed to normalize z-scores, provide gray-level discretization, and voxel size resampling.

Statistical analysis. Statistical analyses were performed by using SAS/STAT version 14.3 (SAS Institute Inc., Cary, NC, United States). Continuous variables were shown as means±standard deviation (SD) and compared with the Mann Whitney U test. Categorical variables were compared with the chi-square test. For all comparisons, a p-value of <0.05 was considered to be statistically significant. Univariate analysis was conducted to extract radiomics features which could discriminate between benign and malignant IPMN. Receiver Operating Characteristic (ROC) analysis was performed to determine the area under the curve (AUC) to evaluate the utility of the features.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the methods described herein. These examples should not be construed as limiting. All percentages are by weight and all solvent mixture proportions are by volume unless otherwise noted.

### Example 1—Identification of MRI-Based Radiomic Features that Distinguish Malignant Cystic Pancreatic Lesions from Benign Lesions

Forty-three patients were included in this preliminary study cohort, including 22 males (range 51-84 years and mean 70.3±10.5 years) and 21 females (range 45-83 years and mean 71±9.3 years). Fifteen subjects (35%) were found to have low or moderate grade dysplasia, while twenty-eight subjects (65%) had high grade dysplasia or invasive carcinoma.

Univariate quantitative imaging analysis revealed 14 features on T1 weighted sequences and 10 features on T2 weighted sequences which were able to discriminate malignant from benign lesions (Table 1 and 2, respectively). All features were within the domain of texture analysis, specifically Gray-Level Co-Occurrence Matrix (GLCM) markers. Table 3 lists abbreviations of examples of radiomics features that may be used with the techniques described herein and their corresponding full names.

TABLE 1
Radiomic features on T1 weighted MRI predictive of
malignancy in the IPMN cohort

| | | std | 95% CI | 95% CI | p |
|---|---|---|---|---|---|
| Radiomics Feature | AUC | error | lower | upper | value |
| GLCM_IBSI_JOINT_AVERAGE_2DF_MR | 0.751 | 0.076 | 0.602 | 0.9 | 0.025 |
| GLCM_IBSI_JOINT_AVERAGE_2DS_MR | 0.751 | 0.076 | 0.602 | 0.9 | 0.025 |
| GLCM_IBSI_SUM_AVERAGE_2DF_MR | 0.751 | 0.076 | 0.602 | 0.9 | 0.025 |
| GLCM_IBSI_SUM_AVERAGE_2DS_MR | 0.751 | 0.076 | 0.602 | 0.9 | 0.025 |
| GLCM_IBSI_AUTOCORR_2DF_MR | 0.743 | 0.077 | 0.592 | 0.894 | 0.029 |
| GLCM_IBSI_AUTOCORR_2DS_MR | 0.743 | 0.077 | 0.592 | 0.894 | 0.029 |
| GLCM_IBSI_JOINT_AVERAGE_2DV_MR | 0.724 | 0.079 | 0.569 | 0.879 | 0.045 |
| GLCM_IBSI_SUM_AVERAGE_2DV_MR | 0.724 | 0.079 | 0.569 | 0.879 | 0.045 |
| SKEWNESS_VOXELS | 0.739 | 0.079 | 0.585 | 0.894 | 0.032 |
| GLCM_IBSI_CLUSTERSHADE_2DV_MR | 0.766 | 0.077 | 0.615 | 0.918 | 0.017 |
| GLCM_IBSI_CLUSTERSHADE_3DV_MR | 0.774 | 0.077 | 0.622 | 0.926 | 0.014 |
| GLCM_IBSI_CLUSTERSHADE_3DF_MR | 0.789 | 0.074 | 0.643 | 0.935 | 0.01 |
| GLCM_IBSI_CLUSTERSHADE_2DF_MR | 0.797 | 0.073 | 0.654 | 0.940 | 0.008 |
| GLCM_IBSI_CLUSTERSHADE_2DS_MR | 0.805 | 0.072 | 0.663 | 0.946 | 0.006 |

AUC-area under the curve;
std-standard;
CI-confidence interval;
GLCM, gray level co-occurrence matrix;
IBSI-image biomarker standardization initiative;
MR-magnetic resonance

TABLE 2
Radiomic features on T2 weighted MRI predictive of
malignancy in the IPMN cohort

| | | std | 95% CI | 95% CI | P |
|---|---|---|---|---|---|
| Radiomics Feature | AUC | error | lower | upper | value |
| GLCM_IBSI_AUTOCORR_3DV_MR | 0.72 | 0.082 | 0.56 | 0.88 | 0.026 |
| GLCM_IBSI_JOINT_AVERAGE_3DV_MR | 0.718 | 0.082 | 0.558 | 0.878 | 0.028 |
| GLCM_IBSI_SUM_AVERAGE_3DV_MR | 0.718 | 0.082 | 0.558 | 0.878 | 0.028 |
| GLCM_IBSI_AUTOCORR_2DF_MR | 0.71 | 0.08 | 0.552 | 0.867 | 0.035 |
| GLCM_IBSI_AUTOCORR_2DS_MR | 0.71 | 0.08 | 0.552 | 0.867 | 0.035 |
| GLCM_IBSI_JOINT_AVERAGE_3DF_MR | 0.71 | 0.081 | 0.551 | 0.868 | 0.035 |
| GLCM_IBSI_SUM_AVERAGE_3DF MR | 0.71 | 0.081 | 0.551 | 0.868 | 0.035 |
| GLCM_IBSI_JOINT_AVERAGE_2DV_MR | 0.704 | 0.083 | 0.541 | 0.868 | 0.04 |
| GLCM_IBSI_SUM_AVERAGE_2DV_MR | 0.704 | 0.083 | 0.541 | 0.868 | 0.04 |
| GLCM_IBSI_AUTOCORR_2DV_MR | 0.702 | 0.083 | 0.54 | 0.864 | 0.042 |

AUC-area under the curve;
std-standard;
CI-confidence interval;
GLCM, gray level co-occurrence matrix;
IBSI-image biomarker standardization initiative;
MR-magnetic resonance

TABLE 3
Radiomics Feature Abbreviations and Full Names

| Radiomics Feature Abbreviations | Radiomics Feature Full Names |
|---|---|
| GLCM_IBSI_INVDIFFNORM_2DF_MR | gray level cooccurence matrix Inverse Difference |
| | Normalized for Grey Leveled Image from image |
| | biomarker standardisation initiative by Slice without |
| | Merging |
| GLCM_IBSI_INVDIFFNORM_2DS_MR | gray level cooccurence matrix Inverse Difference |
| | Normalized for Grey Leveled Image from image |
| | biomarker standardisation initiative by Slice with |
| | Merging by Slice |
| GLCM_IBSI_SUM_AVERAGE_2DF_MR | gray level cooccurence matrix Sum Average for |
| | Grey Leveled Image from image biomarker |
| | standardisation initiative by Slice without Merging |
| GLCM_IBSI_SUM_AVERAGE_2DS_MR | gray level cooccurence matrix Sum Average for |
| | Grey Leveled Image from image biomarker |
| | standardisation initiative by Slice with Merging by |
| | Slice |
| GLCM_IBSI_AUTOCORR_2DF_MR | gray level cooccurence matrix Autocorrelation for |
| | Grey Leveled Image from image biomarker |
| | standardisation initiative by Slice without Merging |
| GLCM_IBSI_AUTOCORR_2DS_MR | gray level cooccurence matrix Autocorrelation for |
| | Grey Leveled Image from image biomarker |
| | standardisation initiative by Slice with Merging by |
| | Slice |
| GLCM_IBSI_JOINT_AVERAGE_2DV_MR | gray level cooccurence matrix Joint Average for |
| | Grey Leveled Image from image biomarker |
| | standardisation initiative by Slice with Full Merging |
| GLCM_IBSI_SUM_AVERAGE_2DV_MR | gray level cooccurence matrix Sum Average for |
| | Grey Leveled Image from image biomarker |
| | standardisation initiative by Slice with Full Merging |
| SKEWNESS_VOXELS | SKEWNESS_VOXELS |
| GLCM_IBSI_CLUSTERSHADE_2DV_MR | gray level cooccurence matrix Cluster Shade for |
| | Grey Leveled image from image biomarker |
| | standardisation initiative by Slice with Merging |
| GLCM_IBSI_CLUSTERSHADE_3DV_MR | gray level cooccurence matrix Cluster Shade for |
| | Grey Leveled image from image biomarker |
| | standardisation initiative by Volume with Full |
| | Merging |
| GLCM_IBSI_CLUSTERSHADE_3DF_MR | gray level cooccurence matrix Cluster Shade for |
| | Grey Leveled image from image biomarker |
| | standardisation initiative by Volume without Merging |
| GLCM_IBSI_CLUSTERSHADE_2DF_MR | gray level cooccurence matrix Cluster Shade for |
| | Grey Leveled image from image biomarker |
| | standardisation initiative by Slice without Merging |
| GLCM_IBSI_CLUSTERSHADE_2DS_MR | gray level cooccurence matrix Cluster Shade for |
| | Grey Leveled image from image biomarker |
| | standardisation initiative by Slice with Merging by |
| | Slice |
| GLCM_IBSI_AUTOCORR_3DV_MR | gray level cooccurence matrix Autocorrelation for |
| | Grey Leveled Image from image biomarker |
| | standardisation initiative by Volume with Full |
| | Merging |
| GLCM_IBSI_JOINT_AVERAGE_3DV_MR | gray level cooccurence matrix Joint Average for |
| | Grey Leveled Image from image biomarker |
| | standardisation initiative by Slice with Full Merging |
| GLCM_IBSI_SUM_AVERAGE_3DV_MR | gray level cooccurence matrix Sum Average for |
| | Grey Leveled Image from image biomarker |
| | standardisation initiative by Slice with Full Merging |
| GLCM_IBSI_AUTOCORR_2DF_MR | gray level cooccurence matrix Autocorrelation for |
| | Grey Leveled Image from image biomarker |
| | standardisation initiative by Slice without Merging |
| GLCM_IBSI_AUTOCORR_2DS_MR | gray level cooccurence matrix Autocorrelation for |
| | Grey Leveled Image from image biomarker |
| | standardisation initiative by Slice with Merging by |
| | Slice |
| GLCM_IBSI_JOINT_AVERAGE_3DF_MR | gray level cooccurence matrix Joint Average for |
| | Grey Leveled Image from image biomarker |
| | standardisation initiative by Volume without Merging |
| GLCM_IBSI_SUM_AVERAGE_3DF_MR | gray level cooccurence matrix Sum Average for |
| | Grey Leveled Image from image biomarker |
| | standardisation initiative by Volume without Merging |
| GLCM_IBSI_JOINT_AVERAGE_2DV_MR | gray level cooccurence matrix Joint Average for |
| | Grey Leveled Image from image biomarker |
| | standardisation initiative by Slice with Full Merging |
| GLCM_IBSI_SUM_AVERAGE_2DV_MR | gray level cooccurence matrix Sum Average for |
| | Grey Leveled Image from image biomarker |
| | standardisation initiative by Slice with Full Merging |
| GLCM_IBSI_AUTOCORR_2DV_MR | gray level cooccurence matrix Autocorrelation for |
| | Grey Leveled Image from image biomarker |
| | standardisation initiative by Slice with Merging |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Gillies R J, Kinahan P E, Hricak H: Radiomics: Images Are More than Pictures, They Are Data. Radiology 2016, 278(2):563-577.
2. Permuth J B, Choi J, Balarunathan Y, Kim J, Chen D T, Chen L, Orcutt S, Doepker M P, Gage K, Zhang G et al: Combining radiomic features with a miRNA classifier may improve prediction of malignant pathology for pancreatic intraductal papillary mucinous neoplasms. Oncotarget 2016, 7(52):85785-85797.
3. Virarkar M, Wong V K, Morani A C, Tamm E P, Bhosale P: Update on quantitative radiomics of pancreatic tumors. Abdominal radiology (New York) 2021.
4. Chakraborty J, Midya A, Gazit L, Attiyeh M, Langdon-Embry L, Allen P J, Do R K G, Simpson A L: CT radiomics to predict high-risk intraductal papillary mucinous neoplasms of the pancreas. Medical physics 2018, 45(11):5019-5029.
5. Cui S, Tang T, Su Q, Wang Y, Shu Z, Yang W, Gong X: Radiomic nomogram based on MRI to predict grade of branching type intraductal papillary mucinous neoplasms of the pancreas: a multicenter study. Cancer imaging: the official publication of the International Cancer Imaging Society 2021, 21(1):26.
6. Harrington K A, Williams T L, Lawrence S A, Chakraborty J, Al Efishat M A, Attiyeh M A, Askan G, Chou Y, Pulvirenti A, McIntyre C A et al: Multimodal radiomics and cyst fluid inflammatory markers model to predict preoperative risk in intraductal papillary mucinous neoplasms. Journal of medical imaging (Bellingham, Wash) 2020, 7(3):031507.
7. Tobaly D, Santinha J, Sartoris R, Dioguardi Burgio M, Matos C, Cros J, Couvelard A, Rebours V, Sauvanet A, Ronot M et al: CT-Based Radiomics Analysis to Predict Malignancy in Patients with Intraductal Papillary Mucinous Neoplasm (IPMN) of the Pancreas. Cancers 2020, 12(11).
8. Hanania A N, Bantis L E, Feng Z, Wang H, Tamm E P, Katz M H, Maitra A, Koay E J: Quantitative imaging to evaluate malignant potential of IPMNs. Oncotarget 2016, 7(52):85776-85784.
9. Polk S L, Choi J W, McGettigan M J, Rose T, Ahmed A, Kim J, Jiang K, Balagurunathan Y, Qi J, Farah P T et al: Multiphase computed tomography radiomics of pancreatic intraductal papillary mucinous neoplasms to predict malignancy. World journal of gastroenterology 2020, 26(24):3458-3471.
10. Tanaka M, Fernández-Del Castillo C, Kamisawa T, Jang J Y, Levy P, Ohtsuka T, Salvia R, Shimizu Y, Tada M, Wolfgang C L: Revisions of international consensus Fukuoka guidelines for the management of IPMN of the pancreas. Pancreatology: official journal of the International Association of Pancreatology (IAP) [et al]2017, 17(5):738-753.
11. Srinivasan N, Teo J Y, Chin Y K, Hennedige T, Tan D M, Low A S, Thng C H, Goh B K P: Systematic review of the clinical utility and validity of the Sendai and Fukuoka Consensus Guidelines for the management of intraductal papillary mucinous neoplasms of the pancreas. HPB: the official journal of the International Hepato Pancreato Biliary Association 2018, 20(6):497-504.

1. A method for evaluating the malignant potential of a pancreatic lesion of a subject, the method comprising:
obtaining a magnetic resonance (MR) image of a region of interest (ROI) that includes at least a portion of the pancreatic lesion of the subject;
extracting one or more radiomic features from the ROI of the MR image;
calculating a score from the one or more extracted radiomic features;
comparing the score to a reference value; and
classifying the pancreatic lesion as malignant or benign based on the comparison.

2. The method of claim 1, wherein the pancreatic lesion is an intraductal papillary mucinous neoplasm (IPMN).

3. The method of claim 1, wherein the one or more radiomic features comprises at least one of one or more T1 weighted radiomic features and one or more T2 weighted radiomic features.

4. (canceled)

5. (canceled)

6. The method of claim 1, wherein the one or more radiomic features comprise at least one feature within the domain of texture analysis and/or at least one gray level co-occurrence matrix (GLCM) feature.

7. (canceled)

8. (canceled)

9. The method of claim 1, wherein the MR image is a set of MR images including: T1W pre-contrast images acquired by gradient echo with fat saturation, or T2W non-fat saturated images acquired by fast spin-echo, or both.

10. The method of claim 1, wherein said extracting of one or more radiomic features includes contouring the ROI on pre-operative abdominal axial T1 weighted fat saturated pre-contrast sequences, or T2 weighted echo-planar fast spin echo (single-shot) sequences, or both.

11. The method of claim 1, wherein said extracting of one or more radiomic features includes performing whole lesion semi-automated tumor segmentation to generate a three-dimensional volume of interest.

12. The method of claim 1, wherein the method further comprises pre-normalization of the MR image, prior to said extracting of one or more radiomic features, to provide one or more of: normalize z-score, provide gray-level discretization, and voxel size resampling.

13. A method for treating an intraductal papillary mucinous neoplasm (IPMN) in a subject, comprising administering a treatment for the IPMN to the subject, wherein the subject has previously been identified as having the IPMN, and wherein the IPMN as been classified as malignant using the method of claim 1.

14. The method of claim 13, wherein the treatment comprises surgical resection, pancreatoduodenectomy (Whipple procedure), chemotherapy, radiation, immunotherapy, or a combination of two or more of the foregoing.

15. (canceled)

16. A non-transitory computer-readable storage device for evaluating the malignant potential of a pancreatic lesion of a subject, the non-transitory computer-readable storage device storing computer executable instructions that, when executed, control a processor to perform operations, the operations including:
accessing a magnetic resonance (MR) image of a region of interest (ROI) that includes at least a portion of the pancreatic lesion of the subject;
extracting one or more radiomic features from the ROI of the MR image;
calculating a score from the one or more extracted radiomic features;
comparing the score to a reference value; and
classifying the pancreatic lesion as malignant or benign based on the comparison.

17. The non-transitory computer-readable storage device of claim 16, wherein said accessing comprises retrieving or otherwise acquiring electronic data from a computer memory, receiving a computer file over a computer network.

18. The non-transitory computer-readable storage device of claim 16, wherein the pancreatic lesion is an intraductal papillary mucinous neoplasm (IPMN).

19. The non-transitory computer-readable storage device of claim 16, wherein the one or more radiomic features comprises at least one of one or more T1 weighted radiomic features and one or more T2 weighted radiomic features.

20. (canceled)

21. The non-transitory computer-readable storage device of claim 16, wherein the one or more radiomic features comprise a plurality of radiomic features including one or more T1 weighted radiomic features, and one or more T2 weighted radiomic features.

22. The non-transitory computer-readable storage device of claim 16, wherein the one or more radiomic features comprise at least one feature within the domain of texture analysis and/or at least one gray level co-occurrence matrix (GLCM) feature.

23. (canceled)

24. (canceled)

25. The non-transitory computer-readable storage device of claim 16, wherein the MR image is a set of MR images including: T1W pre-contrast images acquired by gradient echo with fat saturation, or T2W non-fat saturated images acquired by fast spin-echo, or both.

26. The non-transitory computer-readable storage device of claim 16, wherein said extracting of one or more radiomic features includes contouring the ROI on pre-operative abdominal axial T1 weighted fat saturated pre-contrast sequences, or T2 weighted echo-planar fast spin echo (single-shot) sequences, or both.

27. The non-transitory computer-readable storage device of claim 16, wherein said extracting of one or more radiomic features includes performing whole lesion semi-automated tumor segmentation to generate a three-dimensional volume of interest.

28. The non-transitory computer-readable storage device of claim 16, wherein the pre-normalization of the MR image, prior to said extracting of one or more radiomic features, to provide one or more of: normalize z-score, provide gray-level discretization, and voxel size resampling.
